(19) United States
(12) Patent Application Publication
(10) Pub. No.: US 2010/0285123 A1
(43) Pub. Date: Nov. 11, 2010

(54) Controlled Release Dosage Formulation of Duloxetine

(76) Inventors: Rudresha Korlakunte Virupakshalah Prasad, Gujarat (IN); Prabhakaran Desomayanandam, Gujarat (IN)

Correspondence Address: LADAS & PARRY LLP, 26 WEST 61ST STREET, NEW YORK, NY, 10023, US

(21) Appl. No.: 11/922,683

(22) PCT Filed: Jun. 20, 2006
(86) PCT No.: PCT/IN2006/000209

§ 371 Date: Jan. 6, 2010

(30) Foreign Application Priority Data
Jun. 20, 2005 (IN) 718/MUM/2005

(51) Int. Cl. A61K 31/381; A61K 9/36; A61P 7/00

(52) U.S. Cl. 424/465; 514/438

(57) ABSTRACT

The preset invention provides a controlled release dosage form of duloxetine comprising a homogenous core comprised of duloxetine or its pharmaceutically acceptable salts, pharmaceutically acceptable polymeric carrier, solubility enhancer, a hydrophobic component, a hydrodynamic diffusion enhancer, a viscolyzing agent and pharmaceutically acceptable excipients; a entering coat on said core and a barrier layer between said core and the enteric coat.

### FIELD OF THE INVENTION

The present invention relates to controlled release dosage formulation of duloxetine or its pharmaceutically acceptable salts, solvate or hydrates thereof. The said dosage forms exhibiting controlled or sustained or extended release for once-a-day oral administration, which provides better control of blood plasma levels, thereby providing improved safety or lower incidences of side effects and tolerability than the conventional delayed release formulations of duloxetine which is generally administered twice daily.

### BACKGROUND OF THE INVENTION

Nearly one third of patients abruptly discontinue antidepressant treatment within the first month, and data indicate that as many as 44% of patients discontinue treatment within the first 3 months. Although reasons for the cessation of antidepressants have not been extensively studied in controlled, randomly assigned, clinical trials, one of the most frequently reported barriers to patient adherence with this class of medication is clearly unpleasant side effects. Poor tolerability, particularly in the early stages of treatment, is associated with a high incidence of patient dropouts. Although tremendous advances have been made in the treatment of depression, there is still considerable room for improvement. One of the areas in which antidepressant treatments need to be substantially improved is in tolerability. Poor tolerability, particularly early in the course of therapy, can result in a higher incidence of dropouts, and non-adherence is an impediment toward people attaining lasting remission—the ultimate goal of depression therapy.

The saw-tooth pattern of plasma drug concentrations following oral drug administration is associated with adverse events at maxima (“peaks”) and loss of therapeutic effect at minima (“troughs”) that have led to intolerability or frequent dosing of many antidepressants. As medication non-adherence represents a major determinant of non-response, drug reformulations aimed at reducing the pharmacokinetic inadequacies of orally dosed immediate-release preparations have been actively explored (C. D. Kilts, Potential new drug delivery systems for antidepressants: An overview; J. Clin. Psychiatry, 2003, 64 (Suppl 18), pp. 31-33).

Controlled-release formulations of antidepressant agents have the potential to improve tolerability by reducing adverse effects early in the course of therapy—a critical period of dramatic dropout. By lowering the peak plasma drug concentrations inherent to the immediate release formulations, side effects for controlled-release formulations can frequently be reduced to more acceptable levels.

Although most antidepressants have similar response rates, controlled-release formulations may be viable alternatives for those patients with tolerability problems that are commonly associated with immediate-release antidepressant formulations.

Agents such as venlafaxine XR (U.S. Pat. No. 6,419,958) and Bupropion SR are slowly released over time to decrease dosage requirements and increase safety. Venlafaxine extended-release (XR), Bupropion sustained-release (SR) (U.S. Pat. No. 6,589,553), and Paroxetine controlled-release (CR) (U.S. Pat. No. 6,548,084) are examples of antidepressant formulations that have demonstrated effectiveness in reducing some of the adverse effects associated with antidepressant treatment. Controlled-release Paroxetine (Paroxetine CR) combines slow release with an enteric coating to decrease nausea and improve overall tolerability. The reduction in side effects for patients taking a controlled-release antidepressant formulation may improve adherence and therefore the likelihood of achieving a favorable treatment outcome.

Dosage forms for oral drug-delivery systems, which enable sustained, extended, or prolonged-release, often contain higher doses of a beneficial substance than do immediate-release preparations, and are typically designed to produce more uniform absorption of the beneficial substances delivered therefrom. Such dosage forms are referred to herein collectively as “controlled release” dosage forms.

Various such controlled-release formulations are well known in the art. For example, beneficial substances may be incorporated into a core particle, bead, or tablet, which is coated with a polymer that controls the rate of drug release. Release mechanisms include drug diffusion through a non-porous coating, drug diffusion through a porous coating, osmotic pumping of drug controlled by the influx of water through the coating, extrusion of core contents through delivery ports in the coating by swelling of core excipients, diffusion through matrix, erosion through a matrix or combinations of these mechanisms. Membrane coatings may be porous or nonporous, may contain delivery ports formed during or after the coating procedure, or may be formed in the use environment. Exemplary controlled release delivery systems are described in U.S. Pat. No. 5,616,345, U.S. Pat. No. 5,637,320, U.S. Pat. No. 5,505,962, U.S. Pat. No. 5,354,556, U.S. Pat. No. 5,567,441, U.S. Pat. No. 5,728,402, U.S. Pat. No. 5,458,887, U.S. Pat. No. 5,736,159, U.S. Pat. No. 4,801,461, U.S. Pat. No. 5,718,700, U.S. Pat. No. 5,540,912, U.S. Pat. No. 5,612,059, U.S. Pat. No. 5,698,220, U.S. Pat. No. 4,285,987, U.S. 5,324,280, U.S. Pat. No. 4,851,228, U.S. Pat. No. 4,968,507 and U.S. Pat. No. 5,366,738.

U.S. Pat. No. 6,548,084 describes controlled-release Paroxetine formulation which combines slow release with an enteric coating U.S. Pat. No. 2005/0042277 discloses pharmaceutical dosage form comprising a pharmaceutical active e.g., benzimidazole type compounds along with a disintegrant, a swellable coating surrounding the core; and an enteric coating surrounding the swellable coating.

U.S. Pat. No. 6,482,440 discloses a long acting microparticle formulations comprising an antidepressant compound selected from the group consisting of fluoxetine, paroxetine, to sertraline, nefazodone, venlafaxine, trazodone, mirtazapine, fluvoxamine, or pharmaceutically-acceptable salts of those compounds, long-chain derivatives of those compounds, and mixtures thereof.

Duloxetine hydrochloride is a dually acting serotonin and non-adrenaline reuptake inhibitor that is approved for the treatment of major depressive disorder, stress urinary incontinence in women and treatment of painful dialectic peripheral neuropathy. Duloxetine delayed-release capsules are approved for use in major depressive disorder and diabetic neuropathic pain in US and Europe and additionally also for urinary incontinence in Europe

However, it is a matter of concern that the conventional immediate release formulations of Duloxetine hydrochloride (marketed as Cymbalta) are associated with a significantly high risk of adverse effects. Infact, in one such longterm study with Duloxetine hydrochloride in major depression (Study F1J-MC-HMAU available at http://www.lillytrials.com/results files/cymbalta/cymbalta summary 4092.pdf) as many as 17.0% of the patients enrolled in the study (218 of 1279) discontinued due to adverse events with the commonest adverse events being nausea, somnolence & vomiting in these patients. Further, as many as 91.4% of the patients enrolled in this study (1169 of 1279) experienced atleast one or more treatment experienced adverse event during the course of the study. Only 8.6% of the patients enrolled in this study (110 of 1279) did not experience any treatment experienced related adverse event during the entire course of the study.

Again, nausea happened to be the most commonly reported treatment experienced adverse event in these patients being reported in almost ⅓^{rd} of the patients—34.0% to be precise (435 of 1279 patients) followed by insomnia in 31.3% patients (400 of 1279) and headache in 30.4% patients (389 of 1279)

Similar such high incidence of adverse events with the conventional immediate release formulations of Duloxetine hydrochloride have been reported in other clinical trials also [(a) Safety and tolerability of duloxetine in the treatment of major depressive disorder: analysis of pooled data from eight placebo-controlled clinical trials; Hudson J I, Wohlreich M M, Kajdasz D K, Mallinckrodt C H, Watkin J G, Martynov O V, Hum Psychopharmacol. 2005 May 24; (b) Incidence and duration of antidepressant-induced nausea: duloxetine compared with paroxetine and Fluoxetine, Greist J, McNamara R K, Mallinckrodt C H, Rayamajhi J N, Raskin, J. Clin Ther., 2004 September; 26(9): 1446-55; (c) Efficacy, safety and tolerability of duloxetine 60 mg once daily in major depression. Cowen P J, Ogilvie A D, Gama J., Curr Med Res Opin. 2005 March; 21(3):345-56; (d) Efficacy and tolerability of Duloxetine, a novel dual reuptake inhibitor, in the treatment of major depressive disorder. Schatzberg A F, J Clin Psychiatry. 2003;64 Suppl 13:30-7]

In fact, in a comparative clinical trial of Duloxetine and Paroxetine in the acute treatment of major depression (Study F1J-MC-HMAT available at http://www.lillytrials.com/results files/cymbalta/cymbalta summary 4091 b.pdf), the incidence of nausea in patients on Duloxetine was 22.1% to 25.3% while it was reported to be 16.1% in patients on Paroxetine and 22% in patients on placebo, headache was reported in 14.0% to 18.7% in patients on Duloxetine, while it was reported in 11.5% in patients on Paroxetine and 11.2% in patients on placebo and insomnia was reported in 17.4% to 19.8% in patients on Duloxetine, while it was reported in 8.0% in patients on Paroxetine and 5.6% in patients on placebo.

The recommended dose of duloxetine capsules is 40 mg/day (given as 20 mg BID) to 60 mg/day (given either once a day or as 30 mg BID) without regard to meals. There is no evidence that doses greater than 60 mg/day confer any additional benefits.

Duloxetine is unstable in acidic environment (gastric pH). In therapeutic dosing with duloxetine capsules, the drug release is delayed for two to three hours due to enteric coating.

The present invention provides controlled release dosage formulations of duloxetine or its pharmaceutically acceptable salts, solvate or hydrates thereof. The said dosage forms exhibiting controlled or sustained, or extended release to be administered once-a-day, orally.

### SUMMARY OF THE INVENTION

In an embodiment of the present invention, there is provided a controlled release dosage forms of duloxetine or its pharmaceutically acceptable salts, solvate or hydrates thereof for once-a-day oral administration, the said dosage forms exhibiting controlled or sustained or extended release profile.

According to another embodiment, the present invention discloses controlled release dosage forms of duloxetine or its pharmaceutically acceptable salts, solvate or hydrates thereof, the said dosage forms exhibiting controlled or sustained or extended release, wherein a smoothened drug plasma concentration to time profile may be obtained, thereby affording a tighter plasma therapeutic range control than can be obtained with multiple daily dosing. In other words, this invention provides a method for eliminating the sharp peaks and troughs (hills and valleys) in blood plasma drug levels induced by multiple daily dosing with conventional delayed release duloxetine formulations.

Further embodiment of the present invention discloses controlled release dosage forms of duloxetine or its pharmaceutically acceptable salts; solvate or hydrates thereof with a better safety profile and tolerability than the conventional delayed release formulations.

In another embodiment of the present invention is disclosed controlled release dosage forms of duloxetine or its pharmaceutically acceptable salts to be administered once-a-day, which may lead to better patient compliance.

### DESCRIPTION OF THE INVENTION

The present invention relates to controlled release dosage forms of duloxetine or its pharmaceutically acceptable salts administered once-a-day, the said dosage forms exhibiting controlled or sustained or extended release which provides better control of blood plasma levels, thereby providing improved safety and tolerability or lower incidences of side effects than the conventional delayed release formulations which is generally administered two times a day.

According to the present invention, duloxetine as used herein can be also in form of a salt such as acid addition salts like hydrochloric, hydrobromic, sulfuric, nitric, and phosphoric acid; or with an organic acid selected from acetic, propanoic, hydroxyacetic, lactic, pyruvic, oxalic, malonic, succinic, maleic, fumaric, malic, tartaric, citric, methanesulfonic, ethanesulfonic, benzenesulfonic, p-toluenesulfonic, cyclamic, salicylic, p-aminosalicylic, and pamoic acid, preferably its salt with hydrochloric acid.

According to the present invention, controlled release dosage form of duloxetine comprises duloxetine or its pharmaceutically acceptable salts, pharmaceutically acceptable polymeric carrier, solubility enhancer. The controlled release dosage form of duloxetine optionally further comprises a hydrophobic component, a hydrodynamic diffusion enhancer, a viscolyzing agent and pharmaceutically accepted excipients. The controlled release dosage form is further coated with entering coating material. The controlled release dosage form of the present invention optionally comprises barrier layer between the core containing duloxetine and the enteric layer.

In the preferred embodiment of the present invention, the controlled release formulation of the present invention comprises a homogenous core surrounded by an enteric coating.

The homogeneous core comprises of,
1. Duloxetine or a pharmaceutically acceptable salt thereof, preferably duloxetine hydrochloride,
2. A pharmaceutically acceptable polymeric carrier (s) or mixture of polymers as carrier,
3. A solubility enhancer or mixture of solubility enhancers,
4. Optionally, a hydrophobic component,
5. Optionally; a hydrodynamic diffusion enhancer,
6. Optionally, a viscolyzing agent,
7. Optionally, other pharmaceutically accepted excipients such as binders, fillers or diluents and lubricants.

A barrier layer between the core containing duloxetine and the enteric layer although not required, but is preferred in the formulation The functions of the separating layer, if required are to provide a smooth base for the application of the enteric layer, to prolong the tablets resistance to acid conditions, to improve stability by inhibiting any interaction between the drug and the enteric polymer in the enteric layer and to improve stability by protecting the drug from light exposure using suitable agents (such as opacifying agents and the like). The barrier layer keeps the core and the enteric layer from coining into direct contact with each other. The barrier layer may also be used to act as a diffusional barrier to migrating core or enteric layer components dissolved in the product mixture.

It has been reported that, when a pharmaceutically acceptable sugar is added to the separating layer, the resistance to acid conditions is markedly increased (U.S. Pat. No. 5,508,276). Accordingly, such a sugar may be included in the separating layer applied to the tablets, either by dissolving as a part of the barrier coating mixture or as a powder.

In general, the barrier layer is composed of coherent or polymeric materials, and finely powdered solid excipients, which constitute fillers. The amount of sugar in the separating layer may be in the range of from 2% to about 10% by weight of the dosage forms.

When a sugar is used at all, and the amount of polymeric or other sticky material may be in the range of from about 0.1 to about 5%. The amount of filler, such as talc, should be in the range of from about 5 to about 15%, based on final product weight.

The enteric coating material surrounding the core comprises admixture of the any of is following components:
1. An enteric polymer or mixture of enteric polymers, which are acid resistant but soluble in intestinal pH.
2. A plasticizer or a mixture of plasticizers.
3. Colorants,
4. Opacifiers and
5. Antiadherants

The controlled release dosage form of duloxetine of the present invention does not release the release the drug in stomach and gastric fluid cannot enter inside the core due to enteric coating. Therefore, no drug release is anticipated in stomach.

Once the dosage form reaches the intestine and pH approaches more than 5.5, the enteric coating starts to dissolve and drug core in polymeric carrier is exposed to the intestinal fluid. The dosage form starts to release the drug in the intestine in a controlled rate by polymeric diffusion, dissolution and erosion mechanisms and the process occurs for prolonged period of time. The dosage form completely erodes and dissolves within the dosing interval (24 h), thereby ensuring complete drug release in the intestine.

The present invention is not restricted to particular component or concentration of the ingredients used in the drug delivery system.

According to the present invention, polymeric carriers may be homopolysaccharide or a heteropolysaccharide, preferably selected from the group consisting of xanthan gum, locust bean gum, propylene glycol ester, galactomannan, glucomannan, guar gum, gum acacia, gum tragacanth, alkali metal carageenates, alginates, cellulose alkyl carboxylates, carboxymethyl cellulose, carboxyethyl cellulose, alkali metal salts of cellulose alkyl carboxylates, sodium carboxymethyl cellulose, carboxypolymethylene, hydroxypropyl methylcelluloses, hydroxypropyl cellulose, hydroxyethyl cellulose, hydroxyethyl methyl cellulose, polyethylene glycols and polyethylene oxides, gellan gum, alginate salts, natural polysaccharides, gum arabica and combinations thereof; hydroxypropylmethyl-cellulose phthalate and the like or their mixtures thereof; ethylene/vinyl alcohol copolymer; ethylene maleic anhydride copolymer; polylactones such as poly(caprolactone) and the like; polyanhydrides such as poly[bis-(p-carboxyphenoxy)propane anhydride], poly(terephthalic acid anhydride) and the like or their mixtures thereof; polyvinyl pyrrolidone; polyesters such as polylactides, polyglycolides, poly(betahydroxybutyric acid) and the like, polyamides and polypeptides such as polylysine, polyglutamic acid and the like; polyethylene glycols and polyethylene oxides commercially available are those under the trade name Polyox®. (Union Carbide, Danbury, Conn.).

According to the present invention, solubility enhancer is solubilizing agents may be selected from (i) agents that inhibit crystal formation of the pharmaceutical or otherwise acts by complexation therewith; (ii) a high HLB (hydrophilic-lipophilic balance) micelle-forming surfactant, particularly anionic surfactants; (iii) citrate esters; (iv) stearate salts; or combinations thereof, particularly combinations of complexation agents with anionic surfactants. Examples of the agents that inhibit crystal formation of the pharmaceutical or otherwise acts by complexation therewith include polyvinylpyrrolidone, polyethylene glycol (particularly PEG 8000), alpha, beta or delta cyclodextrins and other modified cyclodextrins, gelatin, maltodextrin, sorbitol, and polyglyceryl mixed vegetable fatty acid esters.

A high HLB, micelle-forming surfactant includes non-ionic and/or anionic surfactants and selected from Tween 20, Tween 60 or Tween 80, Gelucire 44/14, and Labrasol; polyoxyethylene or polyethylene-containing surfactants, or other long chain anionic surfactants, particularly sodium lauryl sulfate or mixtures thereof.

A citrate ester derivatives include alkyl esters, preferably triethyl citrate. A stearate salts include magnesium stearate, sodium stearate, calcium stearate, and zinc stearate. Combinations of these types of non-swelling solubilizing agents are especially effective.

According to the present invention, a hydrophobic component includes waxes, ethyl cellulose, methacrylate polymers, stearates, cellulose esters, cellulose ethers and cellulose ester-ethers. These materials include cellulose acylate, cellulose ethyl ether, cellulose diacylate, cellulose diacylate, cellulose acetate, cellulose diacetate, cellulose diacetate, mono-, di-, or tri-cellulose alkane, mono-, di-, or tricellulose aroyl and the like or their combination.

According to the present invention, a hydrodynamic diffusion enhancer(s) may be selected from the group comprising of gellan gum, starches, clays, celluloses, cellulose derivatives, alginates, crospovidone (Polyplasdone® and Polyplasdone® XL (ISP, Wayne, N.J.)), croscarmellose sodium (Ac-Di-Sol®, FMC Corp., Philadelphia, Pa.), sodium starch glycolate (Explotab,®, Penwest, Patterson, N.Y.) and combinations thereof. Any excipient which has the inherent capability of drawing water towards it, thereby increasing the rate at which water diffuses through a membrane, then absorbs this water, and swells and increases its volume and creates an internal hydrodynamic pressure would be capable of functioning as a hydrodynamic diffusion enhancer and thus would be a suitable hydrodynamic diffusion enhancer for the pharmaceutical compositions of the present invention.

A viscolyzing agent which, upon contact with gastrointestinal fluid, instantaneously viscolyzes to maintain tablet integrity when stirred in an aqueous medium, and in sustaining the release of the drug even in low concentration. Preferably, the viscolyzing agent comprises of a carbohydrate gum. Examples of carbohydrate gums that may be used in the present invention include xanthan gum, tragacanth gum, gum karaya, guar gum, acacia, and the like.

The binder may be any pharmaceutically acceptable film former, which can be utilised to bind the powder mixture together with an adhesive instead of compaction in order to form granules for making compressed tablets. These polymers include polyvinyl pyrrolidone, carboxyvinyl polymer, methyl cellulose, hydroxyethyl cellulose, hydroxypropyl cellulose, hydroxypropylmethyl cellulose, dextrin, maltodextrin and the like or mixtures thereof.

The term “diluent” is intended to mean inert substances used as fillers to create the desired bulk, flow properties and compression characteristics in the preparation of formulations. Such compounds include, but without limitation, dibasic calcium phosphate, kaolin, lactose, starch, sucrose, mannitol, microcrystalline cellulose, powdered cellulose, precipitated calcium carbonate, sorbitol, starch and the like or combinations thereof.

The lubricants and flow promoters are selected from the group comprising of stearic acid, talc, waxes, stearic acid salts, stearic acid derivatives, sodium stearyl fumarate, corn starch, silica derivatives and combinations thereof.

The pH sensitive material or enteric polymers generally do not degrade and begin to release the active drug until a pH above 3.0 is reached and preferably above 5.5. Enteric or pH sensitive polymers that can be used for the enteric or pH sensitive coating of the present invention may be selected from the group comprising of Eudragit to L (poly(methacrylic acid methyl methacrylate) in 1:1 ratio (MW No. Av. 135,000—USP Type A) or Eudragit S (poly(methacrylic acid, methylmethacrylate) in 1:2 ratio (MW No. Av. 135,000—USP Type B), hydroxypropyl methyl cellulose phthalate, cellulose acetate phthalate, polyvinyl acetate phthalate and the like or their mixtures.

Plasticizers that can be used in the invention include all those that are generally incorporated into polymeric coatings of delivery devices. Plasticizers that may be used in the membrane of the present invention may be selected from acetyl triethyl citrate, acetyl tributyl citrate, triethyl citrate, acetylated monoglycerids, glycerol, polyethylene glycol, triacetin, propylene glycol, dibutyl phthalate, diethyl phthalate, isopropyl phthalate, dimethyl phthalate, dactyl phthalate, dibutyl sebacate, dimethyl sebacate, castor oil, glycerol monostearate, fractionated coconut oil, and others used alone or in combination. Suitable plasticizers also include, by way of example and without limitation, low molecular weight polymers, oligomers, copolymers, oils, small organic molecules, low molecular weight polyols having aliphatic hydroxyls, ester-type plasticizers, glycol esters, poly(propylene glycol), multi-block polymers, single-block polymers, low molecular weight poly(ethylene glycol), citrate ester-type plasticizers, triacetin, propylene glycol and glycerin. Such plasticizers can also include ethylene glycol, 1,2-butylene glycol, 2,3-butylene glycol, styrene glycol, diethylene glycol, triethylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutyl sebacate, acetyl tributyl citrate, triethyl citrate, acetyl triethyl citrate, tributyl citrate and allyl glycolate used alone or in combination.

The formulations of the present invention can be in the form of oral dosage forms such as tablets, capsules, pellets, granules, microtablets or minitalbets etc. Preferably, formulation is either compressed into tablets or granulated and filled into capsules

“Mini tablets” are an alternative to pellet-based multiparticulate dosage forms (UK Patent 2:176999) as tablets in the size range of 2 mm to 4 mm diameter. Mini tablets can be prepared by known techniques as reported in prior art. These tablets can be filled into capsules using the same processes and equipment used to fill pellets. In the present study, mini tablets are used to increase the absorption of the drug from the absorption site which may be in the region of the small intestine. The drug when present in mini tablets is administered as multiple unit dosage form in a capsule. The mini tablet consists of a core containing the drug, a barrier coat and an enteric layer as described above.

Controlled release formulation of Duloxetine of the present invention may be characterized by one or more or all of the following characteristics:
1. Provides the same or lower total exposure (AUC₀₋ₜ and AUC O-α) of Duloxetine with a once-daily controlled release dose as the immediate release formulation with single or multiple doses.
2. Slower release in in-vitro dissolution tests at different pH conditions.
3. Slower release in gastrointestinal tract of mammals including human beings. The proposed controlled release formulation of Duloxetine will have lower maximum plasma concentrations (Cₘₐₓ) and would achieve this at a later time (higher Tₘₐₓ) as compared to the immediate release dosage form of Duloxetine (Cymbalta).
4. Compared to the immediate release formulation of Duloxetine, the proposed controlled release formulation would provide lesser degree of fluctuations between maximum (Cₛₛ max) and minimal plasma (Cₛₛ min) concentrations at steady state pharmacokinetics. These fluctuations in Cₛₛ max and Cₛₛ min are commonly referred in literature as well as in the current patent application as hills and valleys, peak and trough etc. The longer apparent elimination half-life of proposed formulation of Duloxetine after single dose would make it suitable for once daily dosing compared with the twice daily dosing regimen required by the current immediate release formulation of Duloxetine including, but not restricted to, 20, 30 & 40 mg strengths of Duloxetine.
5. These differential characteristics listed above would potentially result in an improved and a better tolerability profile compared to the immediate release formulation of Duloxetine (Cymbalta). The increased compliance observed with the proposed controlled release dosage form of Duloxetine could improve the appropriate management of patients in need to treatment with Duloxetine including, but not restricted to, major depressive disorders, major depressive disorder, stress urinary incontinence.

The controlled release dosage forms of duloxetine or its pharmaceutically acceptable salt thereof described in the present invention has the following advantages.
1. It is possible to obtain a smoothened drug plasma concentration to time profile with the controlled release dosage forms of duloxetine, thereby affording a tighter plasma therapeutic range control than can be obtained with multiple daily dosing. In other words, this invention provides a method for eliminating the sharp peaks and troughs (hills and valleys) in blood plasma drug levels induced by multiple daily dosing with conventional delayed release duloxetine hydrochloride tablets.
2. Controlled release dosage forms of duloxetine may lead to better safety profile and tolerability than the conventional delayed release formulations on account of once-a-day administration.
3. Controlled release dosage forms of duloxetine or its pharmaceutically acceptable salt thereof may be administered once-a-day, which may lead to better patient compliance as the conventional delayed release formulations generally administered twice a day.

In the preferred embodiment, the controlled release dosage form of duloxetine contains 10 to 100 mg of Duloxetine hydrochloride.

The controlled release dosage forms of Duloxetine can be prepared by employing any method selected from wet granulation, dry granulation, melt granulation or the method known by the person skilled in the art.

### Process of Preparation

Duloxetine Hydrochloride and other ingredient's were sieved (ASTM#60) separately and mixed by geometric dilution. The mixture was roll compacted and subsequently size reduced by oscillating granulator (ASTM#16). The obtained granules were lubricated with magnesium stearate and talc. Finally, the granules were compressed using suitable punches in a rotary compression machine.

A barrier layer was applied by dissolving the ingredients in isopropylalcohol and water (85:15 ratio) mixture. Talc was dispersed in the solution using a homogenizer. The resulting suspension was sprayed on to the core tablets using Gansons spray pan coating machine.

The enteric coating suspension was prepared by first dissolving enteric coating materials (e.g., HPMC-P 55) in dicholoromethane: isopropyl alcohol (70:30). Talc was added to the above solution and dispersed using a homogenizer. The resulting suspension was sprayed onto the barrier-coated tablets using Gansons spray pan coating machine.

The controlled release dosage forms of Duloxetine and its process described in the present invention is demonstrated in examples illustrated below. These examples are provided as illustration only and therefore should not be construed as limitation of the scope of invention.

### Example 1

TABLE 1

| | Quantity for one tablet |
|---|---|
| Ingredients | (mg) |
| Core composition | |
| Duloxetine HCl | 44.87 |
| H.P.M.C 15 cps | 112.5 |
| H.P.M.C 6 cps | 112.5 |
| Lactose | 41.3 |
| S.L.S | 8.0 |
| Xanthan gum | 2.0 |
| Sodium alginate | 1.0 |
| Magnesium stearate | 1.0 |
| Talc | 2.0 |
| Barrier coating | |
| HPMC 6 cps | 9.15 |
| Enteric coating | |
| Eudragit L 100-55 | 18.52 |
| Purified Talc | 2.52 |
| Triethyl citrate | 4.20 |
| Total | 359.56 |

Dissolution study of the enteric coated tablets was conducted in dissolution media of different pH i.e., 0.1N hydrochloric acid pH 1.2 (0-2 h), and phosphate buffer pH 6.8 (2-20 h), using USP apparatus type 1 (basket) at 100 rpm. The dissolution results are given in Table 2.

TABLE 2
In-vitro dissolution profile of duloxetine
40 mg enteric coated tablets

| Time (h) | Av. % release |
|---|---|
| 0 | 0 |
| 2 | 0 |
| 4 | 9.77 |
| 6 | 37.33 |
| 8 | 61.32 |
| 10 | 78.94 |
| 12 | 88.07 |
| 16 | 90.12 |
| 20 | 92.12 |

### Example 2

TABLE 3

| Ingredients | Quantity for one tablet (mg) |
|---|---|
| Core composition | |
| Duloxetine HCl | 67.305 |
| H.P.M.C. 15 cps | 125.0 |
| H.P.M.C. 6 cps | 125.0 |
| Lactose | 18.695 |
| S.L.S | 8.0 |
| Xanthan gum | 2.0 |
| Sodium alginate | 1.0 |
| Magnesium stearate | 1.0 |
| Talc | 2.0 |
| Barrier coating | |
| HPMC 6 cps | 7.42 |
| Triethyl citrate | 0.82 |
| Enteric coating | |
| Eudragit L100-55 | 17.57 |
| Purified talc | 5.40 |
| Triethyl citrate | 4.05 |
| Total | 385.26 |

The product was made substantially according to the process used in Example 1. The dissolution study was followed as in example 1.

TABLE 4
In-vitro release

| Time (h) | Av. % release |
|---|---|
| 0 | 0 |
| 2 | 0 |
| 4 | 10.04 |
| 6 | 35.00 |
| 8 | 57.39 |
| 10 | 73.58 |
| 12 | 85.19 |
| 16 | 86.66 |
| 20 | 89.94 |

### Example 3

TABLE 5

| Ingredients | Quantity for one tablet (mg) |
|---|---|
| Core composition | |
| Duloxetine HCl | 67.305 |
| H.P.M.C 15 cps | 125.00 |
| H.P.M.C 6 cps | 125.00 |
| Lactose | 18.695 |
| S.L.S | 8.00 |
| Xanthan gum | 2.00 |
| Sodium alginate | 1.00 |
| Magnesium stearate | 2.00 |
| Talc | 1.00 |
| Barrier coating | |
| HPMC P 6 cps | 10.37 |
| Triethyl citrate | 1.15 |
| Enteric coating | |
| HPMC-P-HP-55 | 21.46 |
| Triethyl citrate | 2.38 |
| Total | 385.36 |

The product was made substantially according to the process used in Example 1. The dissolution study was followed as in example 1.

TABLE 6
In-vitro release

| Time (h) | Av. % release |
|---|---|
| 0 | 0 |
| 2 | 0 |
| 4 | 32.6 |
| 6 | 36.3 |
| 8 | 67.1 |
| 10 | 85.3 |
| 12 | 88.1 |
| 16 | 88.7 |
| 20 | 89.5 |

### Example 4

TABLE 7

| Ingredients | Quantity for one tablet (mg) |
|---|---|
| Core composition | |
| Duloxetine HCl | 67.305 |
| H.P.M.C 15 cps | 125.00 |
| H.P.M.C 6 cps | 125.00 |
| Lactose | 18.695 |
| S.L.S | 8.00 |
| Xanthan gum | 2.00 |
| Sodium alginate | 1.00 |
| Magnesium stearate | 2.00 |
| Talc | 1.00 |
| Barrier coating | |
| HPMC-Acetate succinate | 9.09 |
| Triethyl citrate | 1.01 |
| Enteric coating | |
| HPMC-Acetate succinate | 27.55 |
| Triethyl citrate | 3.06 |
| Total | 390.71 |

The product was made substantially according to the process used in Example 1. The dissolution study was followed as in example 1.

TABLE 8
In-vitro release

| Time (h) | Av. % release |
|---|---|
| 0 | 0 |
| 2 | 0 |
| 4 | 19.48 |
| 6 | 41.27 |
| 8 | 64.77 |
| 10 | 80.06 |
| 12 | 83.47 |
| 16 | 84.71 |
| 20 | 86.92 |

### Example 5

TABLE 9

| | Quantity for one tablet |
|---|---|
| Ingredients | (mg) |
| Core composition | |
| Duloxetine HCl | 67.307 |
| H.P.M.C 15 cps | 85.0 |
| H.P.M.C 6 cps | 85.0 |
| Lactose | 8.0 |
| S.L.S | 18.695 |
| Xanthan gum | 2.0 |
| Sodium aginate | 1.0 |
| Magnesium stearate | 2.0 |
| Talc | 1.0 |
| Barrier coating | |
| HPMC 6 cps | 0.95 |
| Sucrose | 1.66 |
| Purified talc | 2.13 |
| Enteric coating | |
| HPMC-P-55 | 10.94 |
| Purified talc | 4.56 |
| Triethyl citrate | 2.73 |
| Total | 290.24 |

The product was made substantially according to the process used in Example 1. The dissolution study was followed as in example 1.

TABLE 10
In-vitro release

| Time (h) | Av. % release |
|---|---|
| 0 | 0 |
| 2 | 0 |
| 4 | 14.7 |
| 6 | 45.2 |
| 8 | 66.25 |
| 10 | 78.65 |
| 12 | 80.85 |
| 16 | 83.69 |
| 20 | 88.52 |

### Example 6

TABLE 11

| | Quantity for one tablet |
|---|---|
| Ingredients | (mg) |
| Core composition | |
| Duloxetine HCl | 67.3075 |
| H.P.M.C.15 cps | 60.0 |
| H.P.M.C 6 cps | 60.0 |
| Lactose | 8.0 |
| S.L.S | 18.695 |
| Xanthan gum | 2.0 |
| Sodium alginate | 1.0 |
| Magnesium stearate | 2.0 |
| Talc | 1.0 |
| Barrier coating | |
| HPMC 6 cps | 0.99 |
| Sucrose | 1.73 |
| Purified talc | 2.23 |
| Enteric coating | |
| HPMC-P-55 | 3.54 |
| Purified talc | 1.48 |
| Triethyl citrate | 0.89 |
| Total | 229.97 |

The product was made substantially according to the process used in Example 1. The dissolution study was followed as in example 1.

TABLE 12
In-vitro release

| Time (h) | Av. % release |
|---|---|
| 0 | 0 |
| 2 | 0 |
| 4 | 15.35 |
| 6 | 43.18 |
| 8 | 62.62 |
| 10 | 75.77 |
| 12 | 81.34 |
| 16 | 88.62 |
| 20 | — |

### Example 7

### Mini Tablets

TABLE 13

| Ingredients | Quantity for one mini tablet (mg) |
|---|---|
| Core composition | |
| Duloxetine HCl | 4.487 |
| H.P.M.C 15 cps | 8.34 |
| H.P.M.C 6 cps | 8.34 |
| Lactose | 1.25 |
| S.L.S | 0.54 |
| Xanthan gum | 0.14 |
| Sodium alginate | 0.067 |
| Magnesium stearate | 0.14 |
| Talc | 0.067 |
| Barrier coating | |
| HPMC 6 cps | 0.42 |
| Triethyl citrate | 0.05 |
| Enteric coating | |
| Eudragit L100-55 | 0.85 |
| Purified talc | 0.26 |
| Triethyl citrate | 0.2 |
| Total | 25.151 |

Mini tablets were prepared as described in example 1 using suitable punches. In vitro release of Duloxetine HCl mini tablets enteric coated (equivalent to 60 mg of duloxetine) were filled in ‘0’ size capsules and subjected to dissolution studies as in example 1.

TABLE 14
In-vitro release

| Time (h) | Av. % release |
|---|---|
| 0 | 0 |
| 1 | 0 |
| 2 | 0 |
| 3 | 7.74 |
| 4 | 34.2 |
| 6 | 63.96 |
| 8 | 79.87 |
| 10 | 81.42 |
| 12 | 84.87 |
| 16 | — |
| 20 | — |

### Example 8

### Mini Tablets

TABLE 15

| Ingredients | Quantity for one mini tablet (mg) |
|---|---|
| Core composition | |
| Duloxetine HCl | 4.487 |
| H.P.M.C 15 cps | 8 |
| H.P.M.C 6 cps | 8 |
| Lactose | 1.130 |
| S.L.S | 0.534 |
| Carbopol 934p | 1 |
| Magnesium stearate | 0.134 |
| Talc | 0.067 |
| Barrier coat | |
| HPMC 6 cps | 0.06 |
| Sucrose | 0.1 |
| Purified talc | 0.13 |
| Enteric coat | |
| HPMC-P-55 | 0.51 |
| Purified talc | 0.21 |
| Triethyl citrate | 0.13 |
| Total | 24.492 |

TABLE 16
In-vitro release

| Time (h) | Av. % release |
|---|---|
| 0 | 0 |
| 2 | 0 |
| 4 | 11.36 |
| 6 | 20.05 |
| 8 | 25.66 |
| 10 | 30.23 |
| 12 | 36.42 |
| 16 | 37.56 |
| 20 | — |

Batches of the coated duloxetine tablets, which have a dissolution rate corresponding to that of Table 4 was taken for bio-study.

Objective of the present study was to compare the single dose oral bioavailability of duloxetine hydrochloride tablet containing 60 mg duloxetine hydrochloride with that of Cymbalta capsule containing 60 mg duloxetine hydrochloride in healthy, adult, male, human subjects under fasting conditions.

An open label, balanced, randomized, two-treatment, two-sequence, two-period, single dose, crossover bioavailability study under fasting condition was carried out.

Six healthy male volunteers were administered with a single tablet containing duloxetine (60 mg)/single capsule of Cymbalta (60 mg). Blood samples were collected at 1, 2, 4, 5, 6, 7, 8, 10, 12, 16, 24, 48, 72 and 96 hrs following drug administration in each period. Drug concentrations in plasma were analyzed using a LC-MS/MS method.

The pharmacokinetic parameters Cₘₐₓ, Tₘₐₓ, AUC₀₋ₜ, AUC_{0-∞}, λ_{z}, t_{1/2}, and AUC_% extrapolated (Residual area) were estimated for duloxetine.

Table 17 shows the pharmacokinetic parameters of duloxetine controlled release tablet and Cymbalta capsule.

TABLE 17

| | Mean ± SD (n = 06) | |
|---|---|---|
| Parameters (Units) | Reference Product(R) | Test Product (T) |
| Mean Tₘₐₓ (h) | 6.000 | 16.000 |
| Mean Cₘₐₓ (ng/ml) | 45.59 | 28.38 |
| AUC₀₋ₜ (ng · hr/mL) | 670.36 | 751.38 |
| AUC_{0-∞} (ng · hr/mL) | 688.28 | 854.38 |

### BRIEF DESCRIPTION OF DRAWINGS

FIG. 1: Linear plot of mean plasma concentration versus time curves of duloxetine after administration of test (T) and reference (R) formulations to healthy, adult, male, human subjects under fasting conditions.

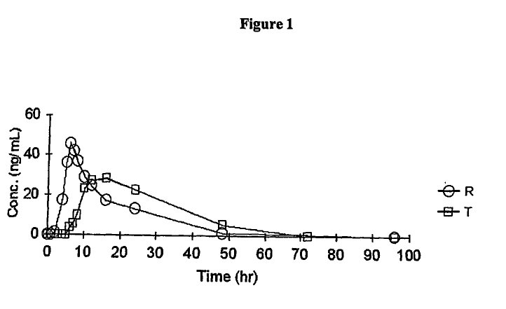

1. A controlled release dosage form of duloxetine for once a day administration, which comprises duloxetine, a pharmaceutically acceptable salt thereof, a pharmaceutically acceptable polymeric carrier and solubility enhancer.

2. The controlled release dosage form of duloxetine according to claim 1, further comprising a hydrophobic component, a hydrodynamic diffusion enhancer, a viscolyzing agent and one or more pharmaceutically acceptable excipients.

3. The controlled release dosage form of duloxetine according to claim 1, coated with enteric coating material.

4. The controlled release dosage form of duloxetine according to claim 1, further comprising a barrier layer between a core containing duloxetine and an enteric layer.

5. The controlled release dosage form of duloxetine as claimed in claim 1 which comprises:
a) a homogenous core comprised of duloxetine or a pharmaceutically acceptable salt thereof, a pharmaceutically acceptable polymeric carrier, solubility enhancer, a hydrophobic component, a hydrodynamic diffusion enhancer, a viscolyzing agent and one or more pharmaceutically acceptable excipients; and
(b) an enteric coating on said core.

6. The controlled release dosage form of duloxetine as claimed in claim 1, wherein said dosage form provides an in vivo plasma profile selected from:
(a) Tmax in a range of from about 5 hrs to about 21 hrs; and
(b) AUC₀₋ₜ in a range from about 70 ng.h/ml to about 900 ng.h/ml.

7. A controlled release dosage form of duloxetine for once a day administration, which comprises duloxetine or it's a pharmaceutically acceptable salt thereof, a pharmaceutically acceptable polymeric carrier and solubility enhancer, wherein said dosage form provides an in vivo plasma profile selected from:
(a) Tmax in a range of from about 5 hrs to about 21 hrs; and
(b) AUC₀₋ₜ in a range from about 70 ng.h/ml to about 900 ng.h/m1.

8. A controlled release dosage form of duloxetine for once a day administration, which comprises duloxetine or it's a pharmaceutically acceptable salt thereof, wherein said dosage form provides an in vivo plasma profile selected from:
(a) Tmax is in range of from about 5 hrs to about 21 hrs; and
(b) AUC₀₋ₜ is in range from about 70 ng.h/ml to about 900 ng.h/ml.

9. The controlled release dosage form of duloxetine according to claim 1, wherein said pharmaceutically acceptable polymeric carrier comprises one or more of homopolysaccharides and heteropolysaccharides.

10. The controlled release dosage form of duloxetine according to claim 1, wherein said solubility enhancer is selected from the group consisting of
(i) agents that inhibit crystal formation of the pharmaceutical or otherwise acts by complexation therewith;
(ii) a high HLB (hydrophilic-lipophilic balance) micelle-forming surfactant;
(iii) citrate esters; and
(iv) stearate salts; or a combinations thereof.

11. The controlled release dosage form of duloxetine according to claim 10, wherein said solubility enhancer is in combinations of complexation agents with anionic surfactants.

12. The controlled release dosage form of duloxetine according claim 10, wherein said solubility enhancer is selected from one or more of inhibitors of crystal formation selected from polyvinylpyrrolidone, polyethylene glycol, cyclodextrins, other modified cyclodextrins, gelatin, maltodextrin, sorbitol, polyglyceryl mixed vegetable fatty acid esters, Tween 20, Tween 60 or Tween 80, Gelucire 44/14, Labrasol, polyoxyethylene, polyethylene-containing surfactants, sodium lauryl sulfate, triethyl citrate, magnesium stearate, sodium stearate, calcium stearate, and zinc stearate.

13. The controlled release dosage form of duloxetine according to claim 2, wherein said hydrophobic component is selected from one or more of waxes, ethyl cellulose, methacrylate polymers, stearates, cellulose esters, cellulose ethers and cellulose ester-ethers.

14. The controlled release dosage form of duloxetine according to claim 2, wherein said hydrodynamic diffusion enhancer is selected from one or more of the group consisting of gellan gum, starches, clays, celluloses, cellulose derivatives, alginates, crospovidone, croscarmellose sodium, and sodium starch glycolate or a combination thereof.

15. The controlled release dosage form of duloxetine according to claim 2, wherein said viscolysing agent is selected from one or more of the carbohydrate gums selected from xanthan gum, tragacanth gum, gum karaya, guar gum, and acacia.

16. The controlled release dosage form of duloxetine according to claim 2, wherein said pharmaceutically acceptable excipients are selected from binders, fillers, diluents and lubricants or a combination thereof.

17. The controlled release dosage form of duloxetine according to claim 16, wherein said binder is selected from one or more of the group consisting of polyvinyl pyrrolidone, carboxyvinyl polymer, methyl cellulose, hydroxyethyl cellulose, hydroxypropyl cellulose, hydroxypropylmethyl cellulose, dextrin, and maltodextrin or a combination thereof.

18. The controlled release dosage form of duloxetine according to claim 16, wherein said filler or diluent is selected from one or more of the group consisting of dibasic calcium phosphate, kaolin, lactose, starch, sucrose, mannitol, microcrystalline cellulose, powdered cellulose, precipitated calcium carbonate, sorbitol, and starch or a combinations thereof.

19. The controlled release dosage form of duloxetine according to claim 16, wherein said lubricant is selected from one or more of the group consisting of stearic acid, talc, waxes, stearic acid salts, stearic acid derivatives, sodium stearyl fumarate, corn starch, and silica derivatives or a combination thereof.

20. The controlled release dosage form of duloxetine according to claim 3, wherein said enteric coat is comprised of an enteric polymer and a plasticizer.

21. The controlled release dosage form of duloxetine according to claim 20, wherein said enteric coat further comprise colorants, opacifiers and antiadherants.

22. The controlled release dosage form of duloxetine according to claim 20, wherein said enteric polymer is selected from one or more of Eudragit L (poly(methacrylic acid methyl methacrylate)), Eudragit S (poly(methacrylic acid, methylmethacrylate)), hydroxypropyl methyl cellulose phthalate, cellulose acetate phthalate, and polyvinyl acetate phthalate or a combination thereof.

23. The controlled release dosage form of duloxetine according to claim 20, wherein said plasticizer is selected from one or more of acetyl triethyl citrate, acetyl tributyl citrate, triethyl citrate, acetylated monoglycerids, glycerol, polyethylene glycol, triacetin, propylene glycol, dibutyl phthalate, diethyl phthalate, isopropyl phthalate, dimethyl phthalate, dactyl phthalate, dibutyl sebacate, dimethyl sebacate, castor oil, glycerol monostearate, and fractionated coconut oil or a combination thereof.

24. The controlled release dosage form of duloxetine according to claim 20, wherein said barrier layer comprises one or more of polymeric materials, fillers, and sugar or a combination thereof.

25. The controlled release dosage form of duloxetine according to claim 24, wherein said barrier layer comprises one or more of hydroxyethyl cellulose, hydroxypropyl cellulose, hydroxypropylmethyl cellulose, talc, and triethyl citrate or a combination thereof.

26. A method for providing therapeutic blood plasma concentration of duloxetine over 24 hour period which comprises administering orally to a patient in need thereof, a controlled release formulation containing duloxetine hydrochloride that provides peak blood plasma levels of duloxetine of no more than about 70 ng/ml.

27. A method of providing a therapeutic blood plasma concentration of duloxetine over a 24 hour period, which comprises administering orally to patient in need thereof, a controlled release dosage form comprising 10 to 100 mg duloxetine hydrochloride that provides a peak blood plasma level of duloxetine in from about 5 to about 21 hours.

28. The controlled release dosage form of duloxetine according to claim 1, wherein said dosage form does not release more than about 60% drug at 6 hours and more than about 90% drug at 8 hours in USP apparatus type 1 (Basket) at 100 RPM using 0.1NHCl for 2 hours followed by buffer of pH 6.8.

29. A controlled release dosage form for oral administration comprising 10 to 100 mg duloxetine hydrochloride, which provides a plasma concentration—time curve ratio of time to mean maximum plasma concentration (Tmax) of duloxetine hydrochloride from about 0.70 to about 1.10 in a patient.

30. A controlled release dosage form for oral administration comprising 10 to 100 mg duloxetine hydrochloride, which provides mean maximum plasma concentration (Cmax) of duloxetine ranging from about 5 ng/ml to about 50 ng/ml in a patient.

31. A controlled release dosage form for oral administration comprising 10 to 100 mg duloxetine hydrochloride, which provides a plasma concentration time curve with an area under the curve ranging from about 70 ng.hr/ml to about 900 ng.hr/ml in a patient.

32. The controlled release dosage according to claim 9, wherein the homopolysaccharide and heteropolysaccharide are selected from the group consisting of xanthan gum, locust bean gum, propylene glycol ester, galactomannan, glucomannan, guar gum, gum acacia, gum tragacanth, alkali metal carageenates, alginates, cellulose alkyl carboxylates, carboxymethyl cellulose, carboxyethyl cellulose, alkali metal salts of cellulose alkyl carboxylates, sodium carboxymethyl cellulose, carboxypolymethylene, hydroxypropyl methylcelluloses, hydroxypropyl cellulose, hydroxyethyl cellulose, hydroxyethyl methyl cellulose, polyethylene glycols and polyethylene oxides, gellan gum, alginate salts, natural polysaccharides, gum arabica, etc. and combinations thereof, hydroxypropylmethyl-cellulose phthalate and the like or their mixtures thereof; ethylene/vinyl alcohol copolymer; ethylene maleic anhydride copolymer; polylactones selected from poly(caprolactone) and the like; polyanhydrides selected from poly[bis-(p-carboxyphenoxy)propane anhydride], poly(terephthalic acid anhydride) and the like or their mixtures thereof; polyvinyl pyrrolidone; polyesters selected from polylactides, polyglycolides, poly(betahydroxybutyric acid) and the like, polyamides and polypeptides selected from polylysine, polyglutamic acid and the like; polyethylene glycols and polyethylene oxides.

33. The controlled release dosage form according to claim 15, wherein the viscolysing agent is selected from one or more of carbohydrate gums selected from xanthan gum, tragacanth gum, gum karaya, guar gum, and acacia.

34. The controlled release dosage from of duloxetine according to claim 13, wherein the cellulose esters, cellulose ethers and cellulose esters-ethers are selected from the group consisting of cellulose acylate, cellulose ethyl ether, cellulose diacylate, cellulose triacylate, cellulose acetate, cellulose diacetate, cellulose triacetate, mono-, di-, or tri-cellulose alkane, and mono-, di-, or tricellulose aroyl or a combination thereof.
